(19) United States
(12) Patent Application Publication
(10) Pub. No.: US 2022/0194598 A1
(43) Pub. Date: Jun. 23, 2022

(54) DATA-DRIVEN MANAGEMENT SYSTEM AND METHOD FOR PASSENGER SAFETY, HEALTH AND COMFORT

(71) Applicant: B/E Aerospace, Inc., Winston Salem, NC (US)

(72) Inventors: Yonghua Zhu, Cork (IE); Matthew Robert Pearson, Hartford, CT (US)

(73) Assignee: B/E Aerospace, Inc., Winston Salem, NC (US)

(21) Appl. No.: 17/557,484

(22) Filed: Dec. 21, 2021

(30) Foreign Application Priority Data
Dec. 23, 2020 (EP) 20217119.5

(51) Int. Cl. B64D 13/06; B64D 11/00

(52) U.S. Cl. CPC B64D 13/06; B64D 11/00; B64D 2013/0625; B64D 2013/0662; B64D 2013/0655

(57) ABSTRACT

A management system (100) for passenger safety, health and comfort, the system comprising a first module (101) configured to collect data from an array of sensors within a passenger cabin, a first set of parameters based on the data collected by the first module, a second set of parameters based on the data collected by the first module, a second module (120) comprising a processor configured to process the first set of parameters and the second set of parameters. The processor is further configured to determine if passenger comfort levels are in the negative and/or if a passenger is showing symptoms of sickness, determine alterations and/or actions to be taken by an environmental control system and/or crew of the passenger cabin to assist in moving the comfort levels to a positive and/or to assist a sick passenger, and alert the crew and/or alter the environmental control system.

### TECHNICAL FIELD

The present invention relates to a data-driven management system and method for passenger safety, health and comfort.

### BACKGROUND

Passenger cabins (e.g. train carriages, aircraft cabins, buses etc.) carry many passengers to/from target destinations. The well-being of the passengers during travel time in terms of safety, health and comfort is an important factor when passengers are travelling. Among all the systems, facilities and technologies for ensuring passengers' safety, health and comfort, particularly, the environmental control systems (ECS) play an important role. For example, in aircraft, the ECS can provide pressurized and conditioned fresh air to the aircraft cabin to ensure the health and comfort of the passengers and crew. However, and in the example of an aircraft, there are numerous complaints from passengers about the uncomfortable environment during the flight, which may include too dry, too hot, too cold, etc. In some examples, there are extreme cases where a person falls ill in a passenger cabin and no prompt help is provided by the crew of the passenger vehicle.

### SUMMARY OF THE INVENTION

In one aspect, there is provided a management system for passenger safety, health and comfort. The system comprises a first module configured to collect data from an array of sensors within a passenger cabin, a first set of parameters based on the data collected by the first module, a second set of parameters based on the data collected by the first module, a second module comprising a processor configured to process the first set of parameters and the second set of parameters. The processor is further configured to determine if passenger comfort levels are in the negative and/or if a passenger is showing symptoms of sickness, determine alterations and/or actions to be taken by an environmental control system and/or crew of the passenger cabin to assist in moving the comfort levels to a positive and/or to assist a sick passenger, and alert the crew and/or alter the environmental control system.

The first set of parameters may be based on static data from the array of sensors. The static data may include temperature, humidity, level of noise, amount of light in the cabin, air velocity and/or air quality.

The second set of parameters may be based on dynamic data from the array of sensors. The dynamic data may include gender of passenger, age of passenger, a determination of whether the passenger is an adult or a child, activity levels of passenger, a perception of whether the passenger is too hot or too cold and/or an analysis of whether the passenger is showing symptoms of sickness.

The first module may be a data acquisition module and the second module may be a decision module. There may also be provided an algorithm module that is configured to receive data from the data acquisition module. The algorithm module may include a processor that processes the dynamic data by image and data processing, integration, fusion and/or analysis to determine the second set of parameters.

The alert may be an audio alert and/or a visual alert.

In another aspect, there is provided a method for passenger safety, health and comfort. The method comprises collecting data from an array of sensors within a passenger cabin with a first module, determining a first set of parameters based on the data collected by the first module, determining a second set of parameters based on the data collected by the first module, analysing the first set of parameters and the second set of parameters with a second module that includes a processor, determining if passenger comfort levels are in the negative and/or if a passenger is showing symptoms of sickness, determining alterations and/or actions to be taken by an environmental control system and/or crew of the passenger cabin to assist in moving the comfort levels to a positive and/or to assist a sick passenger, and alert the crew and/or alter the environmental control system.

The first set of parameters may be based on static data from the array of sensors. The static data may include temperature, humidity, level of noise, amount of light in the cabin, air velocity and/or air quality.

The second set of parameters may be based on dynamic data from the array of sensors. The dynamic data may include gender of passenger, age of passenger, a determination of whether the passenger is an adult or a child, activity levels of passenger, a perception of whether the passenger is too hot or too cold and/or an analysis of whether the passenger is showing symptoms of sickness.

The first module may be a data acquisition module and the second module may be a decision module. There may also be provided an algorithm module that is configured to receive data from the data acquisition module, and wherein the algorithm module may include a processor that processes the dynamic data by image and data processing, integration, fusion and/or analysis to determine the second set of parameters.

### BRIEF DESCRIPTION OF THE DRAWINGS

FIG. 1 shows an example of a management system for passenger safety, health and comfort.

FIG. 2 shows an example of a management method for passenger safety, health and comfort.

### DETAILED DESCRIPTION

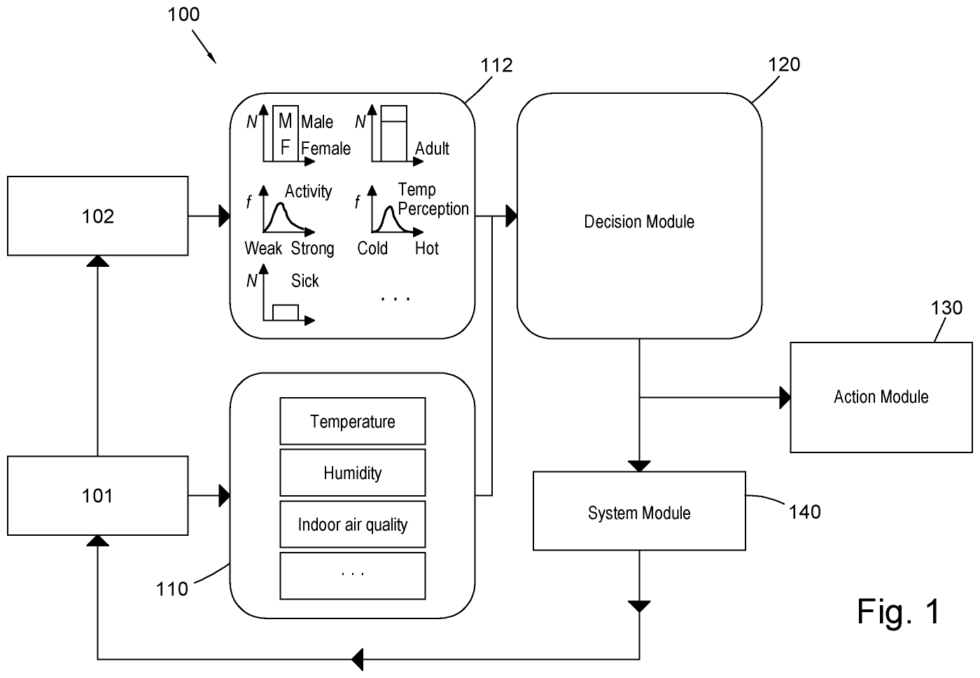

An example of a management system 100 for passenger safety, health and comfort is shown in FIG. 1. The management system 100 may include a data acquisition module 101 that collects information from an array of sensors (not shown) within the passenger cabin. The array of sensors may include video sensing, image sensing, measurement sensing, soft sensing, and the like. The array of sensors are located within the passenger cabin and are utilised to sense various parameters of the environment. The data collected from the data acquisition module 101 may be filtered into data that has fixed values (e.g. static data) and data that needs further processing (e.g. dynamic data). The static data may include values that are provided to the data acquisition module from the environmental control system (ECS), or values from various sensors within the array of sensors in the passenger cabin. For example, the static data may include temperature, humidity and air quality of the air in the passenger cabin. The dynamic data that may need further processing, could include image processing and/or dynamic data retrieved from the various sensors in the array of sensors in the passenger cabin.

The data acquisition module 101 outputs a set of first parameters 110 based on the output of the static data. The first parameters may include, for example, the temperature, humidity, level of noise, amount of light in cabin, air velocity and air quality etc., of the passenger cabin. The data acquisition module 101 relays the dynamic data to an algorithm module 102. The algorithm module may include a processor that processes the dynamic data by image and data processing, integration, fusion and analysis to determine a second set of parameters 112 based on the outputs of the dynamic data after it has been processed. The second parameters may include, for example, the gender of the passengers, whether the passengers are children or adults, estimated ages of the passengers, the activity levels of the passengers, a perception of whether the passengers are too hot or too cold, analysis of whether a passenger is showing symptoms of sickness, etc.

The first set of parameters 110 and the second set of parameters 112 are then output to a decision module 120. The decision module 120 may include one or more processors that process the information received from the first set of parameters 110 and the second set of parameters 120. The decision module 120 will sort through and analyse the data to, for example, determine the comfort level of the passengers and/or to determine if a passenger is showing symptoms of sickness. The comfort level of the passengers is, of course, based on various factors, such as the dryness of the air, humidity, light brightness in the cabin, temperature of the cabin, or the like. The decision module 120 processes the data and determines if the passenger comfort levels are in the positive or in the negative. When the decision module 120 determines that the passenger comfort levels are in the positive, the decision module 120 determines that no further changes to the environment are necessary. When the decision module 120 determines that passenger comfort levels are in the negative, the decision module 120 determines the factors that are contributing to the level of discomfort by analysing the static and dynamic data. Based on this analysis, the decision module 120 determines what alterations need to be made to the cabin environment to raise the passenger comfort levels to the positive. In addition to the above, the decision module 120 may simultaneously determine if a passenger is showing symptoms of sickness by, for example, analysing the video/image sensors for signs of sickness. The decision module 120 would identify the passenger that is displaying symptoms and determine if the passenger is sick or is not sick.

The decision module 120 may then provide outputs based on the analysis of the data. The decision module 120 may filter alerts and actions that need to be taken to an action module 130 that may alert cabin staff to take necessary actions to ensure that the passenger comfort level is raised from negative to positive. Examples of such actions may be to alert cabin staff to provide blankets or water if the temperature is too low or the air is too dry. The decision module 120 may also alert cabin staff to take urgent measures if it is determined that a passenger is sick. Examples of these actions may be to provide medicine, oxygen or request an emergency to the passenger manager on board the vehicle. The alerts may be provided directly to the cabin staff by paging, pinging or alerting on a central computer or tablet—either with visual alerts and/or audible alerts.

In addition to the action module 130, there may be provided a system module 140 that runs additionally or simultaneously with the action module 130. The decision module 120, after processing the data, may output calculations to the system module 140 that do not need to be actioned by cabin staff and can directly be altered in the on-board computers to ensure that passenger comfort levels move from a negative to a positive. As an example, the decision module 120 may determine that the temperature of the cabin is too hot and, therefore, the decision module 120 would then output to the system module 140 that the temperature of the cabin needs to be reduced. The system module 140 then reduces the temperature of the cabin, where appropriate. Likewise, the decision module 120 may determine that the air of the cabin is too dry and, therefore, the decision module 120 instructs the system module 140 to change the humidity of the cabin air. Further examples may include, altering the brightness of the lights in response to the passenger comfort level being in the negative to raise the passenger comfort levels to the positive.

The system as described in FIG. 1 therefore provides a data-driven management system that can ascertain if passengers are feeling comfortable and/or are experiencing symptoms of sickness. Therefore, the passenger well-being is monitored throughout the journey and necessary action is taken by the crew and/or by the environment control system of the passenger vehicle. The combination of static and dynamic data, as well as the processes described above, allow for a more efficient tackling of environmental factors that make the passengers more comfortable during their journey.

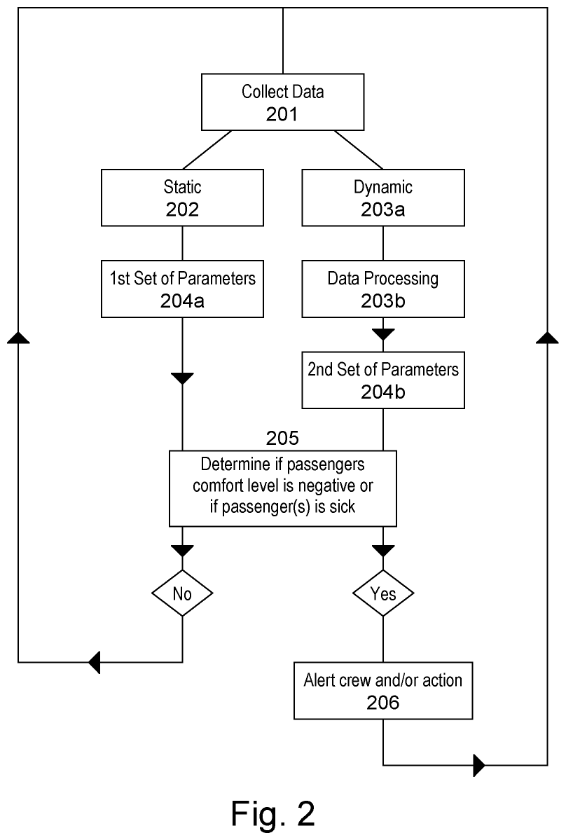

FIG. 2 shows an example of a management method using the system described above for passenger safety, health and comfort.

At step 201, data is collected from the array of sensors as discussed above. The data collected at step 201 is then determined to be static data in step 202 or dynamic data in step 203a. The static data is then processed to determine a first set of parameters at step 204a. The dynamic data is separately processed and analysed in step 203b and then this information is processed to determine a second set of parameters at step 204b. At step 205, the first and second set of parameters are analysed to determine if the passengers comfort level is in the negative and/or whether a passenger is presenting symptoms of sickness. If it is determined that the passenger is uncomfortable and that the passenger comfort levels are in the negative and/or if a passenger is experiencing symptoms of sickness, the method alerts the crew of the cabin and/or provides instructions to the environmental control system to take action in the central computer (e.g. reduce temperature, increase humidity etc.) at step 206. After this step, the method returns to step 201 to continue monitoring of the cabin. If it is determined that the passenger comfort level is in the positive and/or that no passenger is experiencing symptoms of sickness, the method returns to step 201 to continue monitoring.

Although this disclosure has been described in terms of preferred examples, it should be understood that these examples are illustrative only and that the claims are not limited to those examples. Those skilled in the art will be able to make modifications and alternatives in view of the disclosure which are contemplated as falling within the scope of the appended claims.

1. A management system for passenger safety, health and comfort, the system comprising:
a first module configured to collect data from an array of sensors within a passenger cabin;
a first set of parameters based on the data collected by the first module;
a second set of parameters based on the data collected by the first module;
a second module comprising a processor configured to process the first set of parameters and the second set of parameters, wherein the processor is further configured to:
determine if passenger comfort levels are in the negative and/or if a passenger is showing symptoms of sickness;
determine alterations and/or actions to be taken by an environmental control system and/or crew of the passenger cabin to assist in moving the comfort levels to a positive and/or to assist a sick passenger; and
alert the crew and/or alter the environmental control system.

2. The management system of claim 1, wherein the first set of parameters is based on static data from the array of sensors.

3. The management system of claim 2, wherein the static data includes temperature, humidity, level of noise, amount of light in the cabin, air velocity and/or air quality.

4. The management system of claim 1, wherein the second set of parameters is based on dynamic data from the array of sensors.

5. The management system of claim 4, wherein the dynamic data includes gender of passenger, age of passenger, a determination of whether the passenger is an adult or a child, activity levels of passenger, a perception of whether the passenger is too hot or too cold and/or an analysis of whether the passenger is showing symptoms of sickness.

6. The management system of claim 1, wherein the first module is a data acquisition module and the second module is a decision module.

7. The management system of claim 6, wherein an algorithm module is configured to receive data from the data acquisition module, and wherein the algorithm module includes a processor that processes the dynamic data by image and data processing, integration, fusion and/or analysis to determine the second set of parameters.

8. The management system of claim 1, wherein the alert is an audio alert and/or a visual alert.

9. A method for passenger safety, health and comfort, the method comprising:
collecting data from an array of sensors within a passenger cabin with a first module;
determining a first set of parameters based on the data collected by the first module;
determining a second set of parameters based on the data collected by the first module;
analysing the first set of parameters and the second set of parameters with a second module that includes a processor;
determining if passenger comfort levels are in the negative and/or if a passenger is showing symptoms of sickness;
determining alterations and/or actions to be taken by an environmental control system and/or crew of the passenger cabin to assist in moving the comfort levels to a positive and/or to assist a sick passenger; and
alert the crew and/or alter the environmental control system.

10. The method of claim 9, wherein the first set of parameters is based on static data from the array of sensors.

11. The method of claim 10, wherein the static data includes temperature, humidity, level of noise, amount of light in the cabin, air velocity and/or air quality.

12. The method of claim 9, wherein the second set of parameters is based on dynamic data from the array of sensors.

13. The method of claim 12, wherein the dynamic data includes gender of passenger, age of passenger, a determination of whether the passenger is an adult or a child, activity levels of passenger, a perception of whether the passenger is too hot or too cold and/or an analysis of whether the passenger is showing symptoms of sickness.

14. The method of claim 9, wherein the first module is a data acquisition module and the second module is a decision module.

15. The method of claim 14, wherein an algorithm module is configured to receive data from the data acquisition module, and wherein the algorithm module includes a processor that processes the dynamic data by image and data processing, integration, fusion and/or analysis to determine the second set of parameters.
